(19) United States
(12) Patent Application Publication
(10) Pub. No.: US 2019/0083399 A9
(43) Pub. Date: Mar. 21, 2019

(54) DRUG DELIVERY COMPOSITION

(71) Applicants: Isa Odidi, Toronto (CA); Amina Odidi, Toronto (CA)

(72) Inventors: Isa Odidi, Toronto (CA); Amina Odidi, Toronto (CA)

(21) Appl. No.: 12/225,956

(22) PCT Filed: Apr. 3, 2007
(86) PCT No.: PCT/CA2007/000548

§ 371 Date: Mar. 16, 2009

Related U.S. Application Data
(63) Continuation of application No. 11/396,031, filed on Apr. 3, 2006, now abandoned.

(51) Int. Cl. A61K 9/16; A61K 9/28; A61K 9/50; A61K 9/20

(52) U.S. Cl. CPC A61K 9/1652; A61K 9/2846; A61K 9/5084; A61K 9/5078; A61K 9/5047; Y02A 50/411; A61K 9/1611; A61K 9/2081; A61K 9/1676; A61K 9/1635; A61K 9/1617; A61K 9/5026

(57) ABSTRACT

A drug delivery composition that comprises extruded spheroids. The spheroids comprise at least one active pharmaceutical ingredient; at least one extrusion-spheronization aid; at least one superdisintegrant; and at least one glidant, at least one lubricant, and/or at least one oil. The spheroids may also be coated. In a further aspect, a drug delivery composition that comprises coated spheroids that have inert spheroids and at least one coating for the spheroids. The coating comprises at least one active pharmaceutical ingredient and at least one superdisintegrant.

### FIELD OF THE INVENTION

The present invention relates to a drug delivery composition. The present invention also relates to its use and method for making the same.

### BACKGROUND OF THE INVENTION

Many techniques have been used to provide controlled and sustained-release pharmaceutical dosage forms in order to maintain therapeutic serum levels of medicaments and to minimize the effects of missed doses of drugs caused by a lack of patient compliance and the requirement of decreasing side effects of drugs by controlling their blood concentration.

For example, there are extended release tablets which have an osmotically active drug core surrounded by a semipermeable membrane. The semipermeable membrane acts to delimit a reservoir chamber. These tablets function by allowing a fluid, such as gastric or intestinal fluid, to permeate the coating membrane and dissolve the active ingredient so it can be released through a passageway in the coating membrane by osmotic tension or if the active ingredient is insoluble in the permeating fluid, pushed through the passageway by an expanding agent such as a hydrogel. Some representative examples of these osmotic tablet devices can be found in U.S. Pat. Nos. 3,845,770, 3,916,899, 4,034,758, 4,077,407 and 4,783,337. The problem with these devices is that they are tedious and difficult to fabricate. Their efficiency and precision is also in doubt as they have been known to break up prematurely or retain some of the drug content during transit in the gastrointestinal tract, which may lead to less drug being released and delivered by such devices. It is, therefore, not uncommon for such devices to contain an overage of drug of at least 10% to account for such inefficiencies in dose delivery. This practice is not economical and presents a danger, especially if potent drugs are used, as these devices have been known to rupture in transit thus releasing excess dose.

The development of efficacious pharmaceutical compositions for controlled or extended release of active pharmaceutical ingredients is hampered considerably by the fact that current best practices depend mostly on polymeric matrix tablet systems; for example, sustained-release devices, such as tablets coated with a release-controlling coat, matrix tablets comprising water soluble polymeric compounds, matrix tablets comprising wax, matrix tablets comprising water insoluble polymeric compounds and the like. For example, U.S. Pat. No. 3,629,393 (Nakamoto) utilizes a three-component system to provide slow release tablets in which granules of an active ingredient with a hydrophobic salt of a fatty acid and a polymer are combined with granules of a hydrocolloid and a carrier and granules of a carrier and an active or a buffering agent, which are then directly compressed into tablets. U.S. Pat. No. 3,728,445 (Bardani) discloses slow release tablets formed by mixing an active ingredient with a solid sugar excipient, granulating the same by moistening with a cellulose acetate phthalate solution, evaporating the solvent, recovering the granules and compressing under high pressure. U.S. Pat. No. 6,645,528 teaches porous drug matrices and methods of manufacture thereof. Such systems are at a disadvantage because they allow drug delivery via a singular unit. This presents a high risk approach to drug delivery as the single unit may be incapacitated during transit in the gastrointestinal tract or its integrity compromised leading to dose dumping. Furthermore, the singular unit tablet may be excreted intact without drug release.

Therefore, there is a need for drug delivery systems that tend to have more reproducible upper gastrointestinal transit patterns than the singular polymeric matrix tablets.

### SUMMARY OF THE INVENTION

In an aspect, there is provided a drug delivery composition comprising extruded spheroids, the spheroids comprising: at least one active pharmaceutical ingredient; at least one extrusion-spheronization aid; at least one superdisintegrant; and at least one glidant, at least one lubricant, and/or at least one oil.

In another aspect, there is provided a drug delivery composition comprising coated spheroids having inert spheroids and at least one coating for the spheroids, the coating comprising at least one active pharmaceutical ingredient and at least one superdisintegrant.

In a further aspect, there is provided a method for administering the drug delivery composition to a mammal to provide a timed, pulsed, chronotherapeutic, extended or controlled release of said at least one active pharmaceutical ingredient.

In yet a further aspect, there is provided a use of the drug delivery composition in a medicament for providing a mammal with a timed, pulsed, chronotherapeutic, extended or controlled release of said at least one active pharmaceutical ingredient.

In another aspect, there is provided a use of the drug delivery composition for providing a mammal with a timed, pulsed, chronotherapeutic, extended or controlled release of said at least one active pharmaceutical ingredient.

In yet another aspect, there is provided a method for making the drug delivery composition, the method comprising:

combining dry materials of the composition to provide a homogeneous blend;

combining the granules with said at least one glidant, at least one lubricant, and/or at least one oil to provide a wetted mass suitable for extrusion-spheronization; and

extruding the wetted mass to form the spheroids.

In a further aspect, the wetted mass has a plasticity. In yet a further aspect, the wetted mass comprises from about 1:0.7 to about 1:2 of the extrusion aid to said at least one glidant, at least one lubricant, and/or at least one oil.

The novel features of the present invention will become apparent to those of skill in the art upon examination of the following detailed description of the invention. It should be understood, however, that the detailed description of the invention and the specific examples presented, while indicating certain embodiments of the present invention, are provided for illustration purposes only because various changes and modifications within the spirit and scope of the invention will become apparent to those of skill in the art from the detailed description of the invention and claims that follow.

### BRIEF DESCRIPTION OF THE DRAWINGS

Certain embodiments of the present invention will now be described more fully with reference to the accompanying drawings:

FIG. 1 is a dissolution profile for capsules of Example 3; and

FIG. 2 is a dissolution profile for tablets of Example 3.

### DETAILED DESCRIPTION OF THE INVENTION

The present invention is directed to a drug delivery composition and to a method of using and preparing same in order to control the rate and extent of delivery of active pharmaceutical ingredient(s) in mammals.

In one embodiment, the drug delivery composition comprises spheroids. The spheroids comprise at least one active pharmaceutical ingredient; at least one extrusion-spheronization aid; at least one superdisintegrant; and at least one glidant, at least one lubricant, and/or at least one oil.

The spheroids can further comprise at least one carbomer, at least one buffering agent, at least one electrolyte, zein, and/or water. The spheroids of the composition can be made by extrusion, typically, an extrusion-spheronization process.

The spheroids can comprise from about 0.1 wt % to about 80 wt % of at least one active pharmaceutical ingredient, from about 10 wt % to about 90 wt % of at least one extrusion-spheronization aid, from about 0.1 wt % to about 70 wt % of at least one superdisintegrant, from about 0.1 wt % to about 70 wt % of at least one glidant, from about 0.1 wt % to about 70 wt % of at least one lubricant, and from about 0.1 wt % to about 50 wt % of at least one oil. Optionally, the spheroids can further comprise from about 0 wt % to about 50 wt % of at least one carbomer, from about 0 wt % to about 25 wt % of at least one buffering agent, from about 0 wt % to about 55 wt % of at least one electrolyte, from about 0 wt % to about 25 wt % of zein, and/or from about 0 wt % to about 10 wt % of water. These wt % are based on the total weight of the spheroid.

Further embodiments of the spheroids include:

The active pharmaceutical ingredient can also be present of from about 5 wt % to about 70 wt %; about 10 wt % to about 70 wt %; about 20 wt % to about 60 wt %; about 30 wt % to about 60 wt %; or from about 40 wt % to about 60 wt %.

The extrusion-spheronization aid can also be present of from about 10 wt % to about 70 wt %; from about 20 wt % to about 70 wt %; about 30 wt % to about 70 wt %; about 40 wt % to about 70 wt %; about 50 wt % to about 70 wt %; or from about 55 wt % to about 70 wt %.

The superdisintegrant can also be present of from about 2 wt % to about 70 wt %; from about 20 wt % to about 70 wt %; about 30 wt % to about 70 wt %; about 40 wt % to about 70 wt %; about 50 wt % to about 70 wt %; or from about 55 wt % to about 70 wt %.

The glidant can also be present of from about 1 wt % to about 20 wt %; from about 1 wt % to about 15 wt %; from about 2 wt % to about 15 wt %; from about 5 wt % to about 15 wt %; or from about 5 wt % to. about 10 wt %.

The lubricant can also be present of from about 0.5 wt % to about 5 wt %; from about 0.5 wt % to about 4 wt %; from about 0.5 wt % to about 3 wt %; from about 0.5 wt % to about 2 wt %; or from about 1 wt % to about 2 wt %.

The oil can also be present of from about 0.5 wt % to about 5 wt %; from about 0.5 wt % to about 4 wt %; from about 0.5 wt % to about 3 wt %; from about 0.5 wt % to about 2 wt %; or from about 1 wt % to about 2 wt %.

Spheroids of drug delivery compositions tend to have more reproducible upper GI transit patterns than the singular polymeric matrix tablets, for example, if dosing in the fed and fasted states is compared. Since GI transit time is an important parameter relevant to the variability of plasma concentration during drug delivery, this makes the use of multi-particulate drug delivery compositions, such as spheroids, more desirable than singular polymeric matrix systems. The use of a multi-particulate drug delivery composition instead of a singular polymeric matrix tablet is more advantageous since the multi-particulate drug delivery composition can contain a plurality of spheroids containing drugs. Therefore, the loss of integrity of a few spheroids is not going to be statistically significant as compared to the singular polymeric matrix tablet of the prior art. Therefore, the delivery of many therapeutic agents will be most effective when made available as a multi-particulate drug delivery composition.

The spheroids of the drug delivery composition described above can also be coated, for example, with at least one layer of a polymeric film coat; at least one layer of enteric coat; at least one layer of non-enteric coat; and/or at least one layer of semi-permeable membrane coat. Typically, the coating is from about 0.5 wt % to about 50 wt % based on the total weight of the spheroid and coating. More typically, the coating is from about 1 wt % to about 20 wt %, from about 1 wt % to about 10 wt %, from about 1 wt % to about 7 wt %, from about 3.5 wt % to about 7 wt %, from about 3.5 wt % to about 6 wt %, or from about 4 wt % to about 5 wt %. Also, there may be more than one layer of coatings, for example, two to three layers of coatings.

The weight percentages of the components in the coating described herein are based on the weight of the coating.

Any suitable coating may be used for the spheroids of the invention. For example, the coatings can include:

An enteric coating which can comprise at least one enteric material and at least one superdisintegrant. Optionally, the coating further comprises at least one wicking agent, carragenaan, and at least one plasticizer. Typically, the coating comprises from about 10 wt % to about 90 wt % of the enteric material, such as cellulose esters or polymethacrylates; from about 0.5 wt % to about 60 wt % of the superdisintegrant; from about 0 wt % to about 60 wt % of the wicking agent, such as microcrystalline cellulose; from about 0 wt % to about 60 wt % carragenaan and from about 0 wt % to about 25 wt % of the plasticizer, such as polyethylene glycol.

A non-enteric coating which can comprise at least one non-enteric material and at least one superdisintegrant. Optionally, the coating further comprises at least one wicking agent, carragenaan, and at least one plasticizer. Typically, the coating comprises from about 10 wt % to about 90 wt % of the non-enteric material, such as ethylcellulose and/or polyvinylacetate; from about 0.5 wt % to about 60 wt % of the superdisintegrant; from about 0 wt % to about 60 wt % of the wicking agent, such as microcrystalline cellulose; from about 0 wt % to about 60 wt % carragenaan and from about 0 wt % to about 25 wt % of the plasticizer, such as polyethylene glycol.

A semi-permeable membrane coating which can comprise at least one semi-permeable membrane material and at least one superdisintegrant. Optionally, the coating further comprises at least one wicking agent, carragenaan, and at least one plasticizer. Typically, the coating comprises from about 10 wt % to about 90 wt % of the semi-permeable membrane material, such as cellulose acetate phthalate; from about 0.5 wt % to about 60 wt % of the superdisintegrant; from about 0 wt % to about 60 wt % of the wicking agent, such as microcrystalline cellulose; from about 0 wt % to about 60 wt % carragenaan and from about 0 wt % to about 25 wt % of the plasticizer, such as polyethylene glycol.

Further embodiments of the coating composition include:

The enteric material can also be present in the coating of from about 5 wt % to about 90 wt %; from about 10 wt % to about 80 wt %; from about 20 wt % to about 80 wt %; from about 30 wt % to about 70 wt %; or from about 40 wt % to about 70 wt %.

The non-enteric material can also be present in the coating of from about 5 wt % to about 90 wt %; from about 10 wt % to about 80 wt %; from about 20 wt % to about 80 wt %; from about 30 wt % to about 70 wt %; or from about 40 wt % to about 70 wt %.

The semi-permeable membrane material can also be present in the coating of from about 5 wt % to about 90 wt %; from about 10 wt % to about 80 wt %; from about 20 wt % to about 80 wt %; from about 30 wt % to about 70 wt %; or from about 40 wt % to about 70 wt %.

The superdisintegrant can also be present in the coating of from about 0.5 wt % to about 55 wt %; from about 0.5 wt % to about 40 wt %; from about 0.5 wt % to about 30 wt %; from about 1 wt % to about 20 wt %; or from about 10 wt % to about 20 wt %.

The wicking agent can also be present in the coating of from about 0.5 wt % to about 55 wt %; from about 0.5 wt % to about 50 wt %; from about 0.5 wt % to about 40 wt %; from about 5 wt % to about 40 wt %; or from about 20 wt % to about 40 wt %.

Carragenaan can also be present in the coating of from about 0.5 wt % to about 55 wt %; from about 0.5 wt % to about 50 wt %; from about 0.5 wt % to about 40 wt %; from about 5 wt % to about 40 wt %; or from about 20 wt % to about 40 wt %.

The plasticizer can also be present in the coating of from about 0.5 wt % to about 25 wt %; from about 1 wt % to about 20 wt %; from about 5 wt % to about 20 wt %; from about 5 wt % to about 15 wt %; or from about 1 wt % to about 5 wt %.

In a specific embodiment, the coating of the coated spheroids comprises from about 10 wt % to about 90 wt % of the enteric material, such as cellulose esters and/or polymethacrylates; from about 0.5 wt % to about 60 wt % of the superdisintegrant; from about 0.5 wt % to about 60 wt % of the wicking agent, such as microcrystalline cellulose; from about 0 wt % to about 60 wt % carragenaan and from about 0 wt % to about 25 wt % plasticizer, such as polyethylene glycol.

In a further embodiment, the coating of the coated spheroids comprises from about 10 wt % to about 90 wt % of the non-enteric material, such as ethylcellulose and/or polyvinylacetate; from about 0.5 wt % to about 60 wt % of the superdisintegrant; from about 0.5 wt % to about 60 wt % of the wicking agent, such as microcrystalline cellulose; from about 0 wt % to about 60 wt % carragenaan and from about 0 wt % to about 25 wt % plasticizer, such as polyethylene glycol. In still a further embodiment, the coating of the coated spheroids comprises from about 10 wt % to about 90 wt % of the semi-permeable membrane material such as cellulose acetate phthalate; from about 0.5 wt % to about 60 wt % of a superdisintegrant; from about 0.5 wt % to about 60 wt % of a wicking agent, such as microcrystalline cellulose; from about 0 wt % to about 60 wt % carragenaan and from about 0 wt % to about 25 wt % plasticizer, such as polyethylene glycol.

In another embodiment, the coating of the coated spheroids comprises from about 10 wt % to about 90 wt % polyvinylacetate and/or ethylcellulose; from about 0.5 wt % to about 60 wt % of a superdisintegrant; from about 0.5 wt % to about 60 wt % of a wicking agent, such as microcrystalline cellulose; from about 0 wt % to about 60 wt % carragenaan and from about 0 wt % to about 25 wt % plasticizer, such as polyethylene glycol.

The spheroids (e.g. with or without coating) of the composition may be encapsulated (e.g. placed within a capsule) and/or compressed into, for example, tablet(s) and/or caplet(s) and/or combined within a sachet. For example, at least one population of spheroids coated with at least one layer of a polymeric film coat are encapsulated or compressed into at least one tablet. In yet another example, at least one population of spheroids coated with at least one layer of enteric coat are encapsulated or compressed into at least one tablet. In a further example, at least one population of spheroids coated with at least one layer of non-enteric coat are encapsulated or compressed into at least one tablet. In still a further example, at least one population of spheroids coated with at least one layer of semi-permeable membrane coat are encapsulated or compressed into at least one tablet.

The drug delivery composition can also comprise coated inert spheroids. Any suitable coating of the inert spheroids is a coating composition that comprises at least one active pharmaceutical ingredient.

In embodiments, the coating can include at least one active pharmaceutical ingredient and at least one superdisintegrant. Typically, the coating comprises from about 0.1 wt % to about 80 wt % of at least one active pharmaceutical ingredient and from about 0.5 wt % to about 60 wt % of the superdisintegrant. Optionally, the coating can further comprise at least one wicking agent, carragenaan, at least one plasticizer, at least one electrolyte, at least one oil, at least one water soluble gellable polymer, at least one water insoluble organosoluble polymer, at least one glidant, at least one buffering agent, and water. Typically, from about 0 wt % to about 60 wt % of a wicking agent; from about 0 wt % to about 60 wt % carragenaan; from about 0 wt % to about 25 wt % plasticizer, such as polyethylene glycol; from about 0 wt % to about 55 wt % of at least one electrolyte, from about 0 wt % to about 55 wt % of at least one oil, from about 0 wt % to about 50 wt % at least one water soluble gellable polymer, from about 0 wt % to about 50 wt % at least one water insoluble organosoluble polymer, from about 0 wt % to about 25 wt % of at least one glidant, from about 0 wt % to about 25 wt % of at least one buffering agent, and/or from about 0 wt % to about 10 wt % of water.

Further embodiments of the coating composition for the inert spheroids include:

The active pharmaceutical ingredient can also be present in the coating of from about 0.5 wt % to about 90 wt %; from about 10 wt % to about 80 wt %; from about 20 wt % to about 80 wt %; from about 30 wt % to about 70 wt %; or from about 40 wt % to about 70 wt %.

The superdisintegrant can also be present in the coating of from about 0.5 wt % to about 55 wt %; from about 0.5 wt % to about 40 wt %; from about 0.5 wt % to about 30 wt %; from about 1 wt % to about 20 wt %; or from about 10 wt % to about 20 wt %.

The wicking agent can also be present in the coating of from about 0.5 wt % to about 90 wt %; from about 0.5 wt % to about 80 wt %; from about 0.5 wt % to about 70 wt %; from about 5 wt % to about 60 wt %; or from about 20 wt % to about 60 wt %.

Carragenaan can also be present in the coating of from about 0.5 wt % to about 90 wt %; from about 0.5 wt % to about 80 wt %; from about 0.5 wt % to about 70 wt %; from about 5 wt % to about 60 wt %; or from about 20 wt % to about 60 wt %.

The plasticizer can also be present in the coating of from about 0.5 wt % to about 25 wt %; from about 1 wt % to about 20 wt %; from about 5 wt % to about 20 wt %; from about 5 wt % to about 15 wt %; or from about 1 wt % to about 5 wt %.

In a specific embodiment, the coating composition comprises from about 0.1 wt % to about 80 wt % of at least one active pharmaceutical ingredient; from about 0.5 wt % to about 60 wt % of a superdisintegrant; from about 0.5 wt % to about 60 wt % of a wicking agent, such as microcrystalline cellulose and/or pectin; from about 0 wt % to about 60 wt % carragenaan and from about 0 wt % to about 25 wt % plasticizer, such as polyethylene glycol. The coating composition can further comprise from about 0 wt % to about 55 wt % of at least one electrolyte, from about 0 wt % to about 55 wt % of at least one oil, from about 0 wt % to about 90 wt % of at least one plasticizer, from about 0 wt % to about 50 wt % at least one water soluble gellable polymer, from about 0 wt % to about 50 wt % at least one water insoluble organosoluble polymer, from about 0 wt % to about 25 wt % of at least one glidant, from about 0 wt % to about 25 wt % of at least one buffering agent, and/or from about 0 wt % to about 10 wt % of water.

In certain embodiments, there is from about 10 wt % to about 90 wt % of the inert spheroids and from about 0.5 wt % to about 50 wt % of the coating composition. The coating composition comprises from about 0.1 wt % to about 80 wt % of at least one active pharmaceutical ingredient, from about 0.1 wt % to about 50 wt % of at least one superdisintegrant, from about 0.5 wt % to about 90 wt % of a wicking agent, and from about 0.5 wt % to about 90 wt % of carrageenan. Typically, the coating composition comprises from about 0.1 wt % to about 80 wt % of at least one active pharmaceutical ingredient, about 0.1 wt % to about 50 wt % of at least one superdisintegrant, from about 0.5 wt % to about 90 wt % of microcrystalline cellulose, and from about 0.5 wt % to about 90 wt % of carrageenan. Optionally, the coating composition can further comprise from about 0 wt % to about 55 wt % of at least one electrolyte, from about 0 wt % to about 55 wt % of at least one oil, from about 0 wt % to about 90 wt % polyethylene glycol, from about 0 wt % to about 50 wt % hydroxypropylmethyl cellulose, from about 0 wt % to about 50 wt % polyvinyl acetate, from about 0 wt % to about 25 wt % of at least one glidant, from about 0 wt % to about 25 wt % of at least one buffering agent, and/or from about 0 wt % to about 10 wt % of water.

Examples of inert spheroids that may be used are any pharmaceutically acceptable, inert spheroid such as, and without being limited thereto, sugar spheroids, starch spheroids and/or cellulose spheroids.

The spheroids and/or coated spheroids of the present invention can be any suitable size for drug delivery. The spheroids may have a diameter of less than about 6 mm; from about 0.01 mm to about 5.0 mm; or from about 0.15 mm to about 5 mm.

The coating is typically applied to the spheroid to yield a surface area of about 0.1 mg/cm² to about 20 mg/cm².

The drug delivery composition embodiments of the present invention can be used for providing a mammal with a timed, pulsed, chronotherapeutic, extended or controlled release of at least one active pharmaceutical ingredient. The drug delivery composition of the present invention may be in any suitable form that provides release of the spheroids. For example, the composition can be in the form of a tablet or capsule such as, encapsulating (e.g. placed within a capsule) or compressing into a tablet at least one population of spheroids. The tablets or capsules themselves can also be coated, for example, with a polymeric film, such as polymethacrylate copolymers, to provide a timed, pulsed, chronotherapeutic, extended or controlled release of at least one active pharmaceutical ingredient.

In an embodiment, there is provided a method for treating a disease for which at least one active pharmaceutical ingredient in the drug delivery composition is effective. The method comprises administering to a mammal in need of such treatment the timed, pulsed, chronotherapeutic, controlled or extended release drug delivery composition of the present invention.

The drug delivery composition of the present invention can be used for the treatment of hypertension, angina, diabetes, HIV AIDS, pain, depression, psychosis, microbial infections, gastro esophageal reflux disease, impotence, cancer, cardiovascular diseases, gastric/stomach ulcers, blood disorders, nausea, epilepsy, Parkinson's disease, obesity, malaria, gout, asthma, erectile dysfunction, impotence, urinary incontinence, irritable bowel syndrome, ulcerative colitis, smoking, arthritis, rhinitis, Alzheimer's disease, attention deficit disorder, cystic fibrosis, anxiety, insomnia, headache, fungal infection, herpes, hyperglycemia, hyperlipidemia, hypotension, high cholesterol, hypothyroidism, infection, inflammation, mania, menopause, multiple sclerosis, osteoporosis, transplant rejection, schizophrenia, neurological disorders.

The drug delivery composition can dissolve rapidly, instantaneously or melt in the mouth, releasing the spheroids. In a specific embodiment, the drug delivery composition has a dissolution profile wherein from about 0% to 50% of active pharmaceutical ingredient(s) is released in the first hour and greater than about 70% is released in approximately 24 hours.

For various rates of release, various populations of spheroids may be used. For example, to obtain pulsed release, a coated population of spheroids can be combined with an uncoated population of spheroids and encapsulated in a capsule or compressed into a tablet. Alternatively, coated spheroids with different release rates can be combined together and encapsulated in a capsule or compressed into a tablet.

### Method of Making Drug Delivery Composition

The spheroids can be prepared by extrusion-spheronization. In addition, drug-powder or drug solution layering can be used to coat the spheroids. In such an embodiment, the spheroids themselves can be inert and the coating itself contain the active pharmaceutical ingredient(s).

When preparing the spheroids, including coated spheroids, liquids tend to migrate to the surface of spheroids and induce surface plasticity. At very low levels, the surface moisture contributes to lubrication and enhances spheroid movement. At high levels, and especially at reduced ratios of the extrusion-spheronization aid, the liquid may cause the spheroids to stick to one another and the spheronizer wall. It may also lead to uncontrolled granule growth and wide distribution of particle size and, therefore, the batch may be destroyed. This underscores the relationship that exists between the amount of liquid for lubrication and the production of spheroids that are free from agglomeration. The drug delivery composition of the present invention introduces a high margin of formulation tolerance which brings about a balance between rigidity and plasticity of the spheroids. Using the method described herein, spheroids within a narrow size distribution range can be manufactured conveniently and consistently. This method lowers the chance of material being discarded or reworked after a production run due to a low yield in the required size range.

Good extrudates and spheroids can be obtained from the spheroid compositions described herein, for example, a composition comprising from about 0.1 wt % to about 80 wt % of at least one active pharmaceutical ingredient, from about 10 wt % to about 90 wt % of at least one extrusion-spheronization aid, from about 0.1 wt % to about 70 wt % of at least one superdisintegrant, from about 0.1 wt % to about 70 wt % of at least one glidant, from about 0.1 wt % to about 70 wt % of at least one lubricant, and from about 0.1 wt % to about 50 wt % of at least one oil. Optionally, the spheroids can further comprise from about 0 wt % to about 50 wt % of at least one carbomer, from about 0 wt % to about 25 wt % of at least one buffering agent, from about 0 wt % to about 55 wt % of at least one electrolyte, from about 0 wt % to about 25 wt % of zein, and/or from about 0 wt % to about 10 wt % of water.

In an embodiment, to produce spheroids using extrusion-spheronization, extrudates are prepared by first blending the dry materials of the composition in a planetary mixer for a suitable time to provide a homogeneous blend; typically, for about 5 minutes. The homogeneous blend is granulated for about 5 minutes using at least one glidant, at least one lubricant, and/or at least one oil such as, for example, water, oil and, sometimes, an aqueous solution of plasticizer. The granulation time, end point and amount of granulation liquid is determined by the behavior (e.g. should have a plasticity) of a resultant wetted mass during extrusion-spheronization operation. Typically, from about 1:0.7 to about 1:2 of the extrusion aid to the at least one glidant, at least one lubricant, and/or at least one oil is used to form the resultant wetted mass. For example, from about 100 wt %:70 wt % to about 100 wt %:200 wt % of the extrusion aid to the at least one glidant, at least one lubricant, and/or at least one oil is used to form the resultant wetted mass. The wetted mass is passed through the extruder to form rods. The extrudates are charged onto the spheronizer rotating plate and spun at a predetermined rpm for about 30 seconds to about 5 minutes or for a suitable time to provide spheroids. The spheroids are harvested and dried. In an embodiment, the spheroids are dried to provide spheroids having a water content of less than about 10 wt %. In a specific embodiment, the spheroids are dried at about 40° C. for about 16 hours in a tray drier oven to provide a water content of less than about 10 wt %. The granulation solution serves as binder, and together with lubricants, oils and glidants listed above aid the extrusion-spheronization process.

To coat spheroids, a coating composition such as, and without being limited thereto, a solution, a dispersion or a suspension of the coating composition, is coated onto the spheroids. The spheroids can have no coating or already have at least one coating prior to the coating with the coating composition. The coating composition can be applied using any suitable coating process used in the pharmaceutical industry that substantially maintains the integrity of a majority of the spheroids. For example, a fluid bed, powder layering and/or a centrifugal process may be used. The coating method can be repeated to provide more than one coating layer.

The coating composition can comprise a polymeric film, an enteric material; a non-enteric material; and/or a semi-permeable membrane material. Typically, the resultant coating is from about 0.5 wt % to about 50 wt % based on the total weight of the spheroid and coating.

In a specific embodiment, the coating composition comprises from about 10 wt % to about 90 wt % of the enteric material, such as cellulose esters and/or polymethacrylates; from about 0.5 wt % to about 60 wt % of the superdisintegrant; from about 0.5 wt % to about 60 wt % of the wicking agent, such as microcrystalline cellulose; from about 0 wt % to about 60 wt % carragenaan and from about 0 wt % to about 25 wt % plasticizer, such as polyethylene glycol.

In a further embodiment, the coating composition comprises from about 10 wt % to about 90 wt % of the non-enteric material, such as ethylcellulose and/or polyvinylacetate; from about 0.5 wt % to about 60 wt % of the superdisintegrant; from about 0.5 wt % to about 60 wt % of the wicking agent, such as microcrystalline cellulose; from about 0 wt % to about 60 wt % carragenaan and from about 0 wt % to about 25 wt % plasticizer, such as polyethylene glycol. In still a further embodiment, the coating composition comprises from about 10 wt % to about 90 wt % of the semi-permeable membrane material such as cellulose acetate phthalate; from about 0.5 wt % to about 60 wt % of a superdisintegrant; from about 0.5 wt % to about 60 wt % of a wicking agent, such as microcrystalline cellulose; from about 0 wt % to about 60 wt % carragenaan and from about 0 wt % to about 25 wt % plasticizer, such as polyethylene glycol.

In another embodiment, the coating composition comprises from about 10 wt % to about 90 wt % polyvinylacetate and/or ethylcellulose; from about 0.5 wt % to about 60 wt % of a superdisintegrant; from about 0.5 wt % to about 60 wt % of a wicking agent, such as microcrystalline cellulose; from about 0 wt % to about 60 wt % carragenaan and from about 0 wt % to about 25 wt % plasticizer, such as polyethylene glycol.

To coat an inert spheroid, a similar method as described above can be used. The coating composition comprises from about 0.1 wt % to about 80 wt % of at least one active pharmaceutical ingredient and from about 0.5 wt % to about 60 wt % of the superdisintegrant. Optionally, the coating can further comprise at least one wicking agent, carragenaan, at least one plasticizer, at least one electrolyte, at least one oil, at least one water soluble gellable polymer, at least one water insoluble organosoluble polymer, at least one glidant, at least one buffering agent, and water. For example, a solution, a dispersion or a suspension of the coating composition is coated onto the inert spheroids. The spheroids can have no coating or already have at least one coating prior to coating with the coating composition. The coating composition can be applied using any suitable coating process used in the pharmaceutical industry that substantially maintains the integrity of a majority of the spheroids. For example, a fluid bed, powder layering and/or a centrifugal process may be used. The inert spheroids can be, for example, sugar, starch and/or cellulose spheroids.

In another embodiment, the coating composition can be applied using powder layering in a coating pan. The coating composition is added to the inert spheroids while rotating the coating pan. The solution is evaporated leaving behind layers of active pharmaceutical ingredient(s) surrounding the spheroids.

Once the coated spheroids are formed as described herein, the spheroids can be further coated. The coated spheroids can also be further coated with one or more layers of a polymeric film.

### Example of Components of Drug Delivery Composition

With respect to the active pharmaceutical ingredient, the active pharmaceutical ingredient refers to chemical or biological molecules providing a therapeutic, diagnostic, or prophylactic effect in vivo. Active pharmaceutical ingredients contemplated for use in the compositions described herein include the following categories and examples of drugs and alternative forms of these drugs such as alternative salt forms, free acid forms, free base forms, and hydrates: analgesics/antipyretics (e.g., aspirin, acetaminophen, ibuprofen, naproxen sodium, buprenorphine, propoxyphene hydrochloride, propoxyphene napsylate, meperidine hydrochloride, hydromorphone hydrochloride, morphine, oxycodone, codeine, dihydrocodeine bitartrate, pentazocine, hydrocodone bitartrate, levorphanol, diflunisal, trolamine salicylate, nalbuphine hydrochloride, mefenamic acid, butorphanol, choline salicylate, butalbital, phenyltoloxamine citrate, diphenhydramine citrate, methotrimeprazine, cinnamedrine hydrochloride, and meprobamate); antiasthamatics (e.g., ketotifen and traxanox); antibiotics (e.g., neomycin, streptomycin, chloramphenicol, cephalosporin, ampicillin, penicillin, tetracycline, and ciprofloxacin); antidepressants (e.g., nefopam, oxypertine, doxepin, amoxapine, trazodone, amitriptyline, maprotiline, pheneizine, desipramine, nortriptyline, tranylcypromine, fluoxetine, doxepin, imipramine, imipramine pamoate, isocarboxazid, trimipramine, venlafaxine, paroxetine, and protriptyline); antidiabetics (e.g., sulfonylurea derivatives); antifungal agents (e.g., griseofulvin, amphotericin B, nystatin, and candicidin); antihypertensive agents (e.g., propanolol, propafenone, oxyprenolol, reserpine, trimethaphan, phenoxybenzamine, pargyline hydrochloride, deserpidine, diazoxide, guanethidine monosulfate, minoxidil, rescinnamine, sodium nitroprusside, rauwolfia serpentina, alseroxylon, and phentolamine); anti-inflammatories (e.g., (non-steroidal) indomethacin, flurbiprofen, naproxen, ibuprofen, ramifenazone, piroxicam, (steroidal) cortisone, dexamethasone, fluazacort, celecoxib, rofecoxib, hydrocortisone, prednisolone, and prednisone); antiteoplastics (e.g., cyclophosphamide, actinomycin, bleomycin, daunorubicin, doxorubicin, epirubicin, mitomycin, methotrexate, fluorouracil, carboplatin, carmustine (BCNU), methyl-CCNU, cisplatin, etoposide, camptothecin and derivatives thereof, phenesterine, paclitaxel and derivatives thereof, docetaxel and derivatives thereof, vinblastine, vincristine, tamoxifen, and piposulfan); antianxiety agents (e.g., lorazepam, prazepam, chlordiazepoxide, oxazepam, clorazepate dipotassium, diazepam, hydroxyzine pamoate, hydroxyzine hydrochloride, alprazolam, droperidol, halazepam, chlormezanone, and dantrolene); immunosuppressive agents (e.g., cyclosporine, azathioprine, mizoribine, and FK506 (tacrolimus)); antimigraine agents (e.g., ergotamine, divalproex, isometheptene mucate, and dichloralphenazone); sedatives/hypnotics (e.g., barbiturates such as pentobarbital, pentobarbital, and secobarbital; and benzodiazapines such as flurazepam hydrochloride, triazolam, and midazolam); antianginal agents (e.g., beta-adrenergic blockers; calcium channel blockers such as nisoldipine; and nitrates such as nitroglycerin, isosorbide dinitrate, pentaerythritol tetranitrate, and erythrityl tetranitrate); antipsychotic agents (e.g., haloperidol, loxapine succinate, loxapine hydrochloride, thioridazine, thioridazine hydrochloride, thiothixene, fluphenazine, fluphenazine decanoate, fluphenazine enanthate, trifluoperazine, chlorpromazine, perphenazine, lithium citrate, respiridone, and prochlorperazine); antimanic agents (e.g., lithium carbonate); antiarrhythmics (e.g., bretylium tosylate, esmolol, amiodarone, encainide, digoxin, digitoxin, mexiletine, disopyramide phosphate, procainamide, quinidine sulfate, quinidine gluconate, quinidine polygalacturonate, flecainide acetate, tocainide, and lidocaine); antiarthritic agents (e.g., phenylbutazone, sulindac, penicillamine, salsalate, piroxicam, azathioprine, indomethacin, meclofenamate, gold sodium thiomalate, auranofin, aurothioglucose, and tolmetin sodium); antigout agents (e.g., colchicine, and allopurinol); anticoagulants (e.g., heparin, heparin sodium, and warfarin sodium); thrombolytic agents (e.g., urokinase, streptokinase, and alteplase); antifibriolytic agents (e.g., aminocaproic acid); hemorheologic agents (e.g., pentoxifylline): antiplatelet agents (e.g., aspirin); anticonvulsants (e.g., valproic acid, divalproex sodium, phenyloin, phenyloin sodium, clonazepam, primidone, phenobarbitol, amobarbital sodium, methsuximide, metharbital, mephobarbital, mephenyloin, phensuximide, paramethadione, ethotoin, phenacemide, secobarbitol sodium, clorazepate dipotassium, and trimethadione); antiparkinson agents (e.g., ethosuximide); antihistamines/antipruritics (e.g., hydroxyzine, diphenhydramine, chlorpheniramine, brompheniramine maleate, cyproheptadine hydrochloride, terfenadine, clemastine fumarate, triprolidine, carbinoxamine, diphenylpyraline, phenindamine, azatadine, tripelennamine, dexchlorpheniramine maleate, methdilazine, loratadine, and); agents useful for calcium regulation (e.g., calcitonin, and parathyroid hormone); antibacterial agents (e.g., amikacin sulfate, aztreonam, chloramphenicol, chloramphenicol palmitate, ciprofloxacin, clindamycin, clindamycin palmitate, clindamycin phosphate, metronidazole, metronidazole hydrochloride, gentamicin sulfate, lincomycin hydrochloride, tobramycin sulfate, vancomycin hydrochloride, polymyxin B sulfate, colistimethate sodium, and colistin sulfate); antiviral agents (e.g., interferon alpha, beta or gamma, zidovudine, amantadine hydrochloride, ribavirin, and acyclovir); antimicrobials (e.g., cephalosporins such as cefazolin sodium, cephradine, cefaclor, cephapirin sodium, ceftizoxime sodium, cefoperazone sodium, cefotetan disodium, cefuroxime e azotil, cefotaxime sodium, cefadroxil monohydrate, cephalexin, cephalothin sodium, cephalexin hydrochloride monohydrate, cefamandole nafate, cefoxitin sodium, cefonicid sodium, ceforanide, ceftriaxone sodium, ceftazidime, cefadroxil, cephradine, and cefuroxime sodium; penicillins such as ampicillin, amoxicillin, penicillin G benzathine, cyclacillin, ampicillin sodium, penicillin G potassium, penicillin V potassium, piperacillin sodium, oxacillin sodium, bacampicillin hydrochloride. cloxacillin sodium, ticarcillin disodium, aziocillin sodium, carbenicillin indanyl sodium, penicillin G procaine, methicillin sodium, and nafcillin sodium; erythromycins such as erythromycin ethylsuccinate, erythromycin, erythromycin estolate, erythromycin lactobionate, erythromycin stearate, and erythromycin ethylsuccinate; and tetracyclines such as tetracycline hydrochloride, doxycycline hyclate, and minocycline hydrochloride, azithromycin, clarithromycin) anti-infectives (e.g., GM-CSF); bronchodilators (e.g., sympathomimetics such as epinephrine hydrochloride, metaproterenol sulfate, terbutaline sulfate, isoetharine, isoetharine mesylate, isoetharine hydrochloride, albuterol sulfate, albuterol, bitolterolmesylate, isoproterenol hydrochloride, terbutaline sulfate, epinephrine bitartrate, metaproterenol sulfate, epinephrine, and epinephrine bitartrate; anticholinergic agents such as ipratropium bromide; xanthines such as aminophylline, dyphylline, metaproterenol sulfate, and aminophylline; mast cell stabilizers such as cromolyn sodium; inhalant corticosteroids such as beclomethasone dipropionate (BDP), and beclomethasone dipropionate monohydrate; salbutamol; ipratropium bromide; budesonide; ketotifen; salmeterol; xinafoate; terbutaline sulfate; triamcinolone; theophylline; nedocromil sodium; metaproterenol sulfate; albuterol; flunisolide; fluticasone proprionate, steroidal compounds and hormones (e.g., androgens such as danazol, testosterone cypionate, fluoxymesterone, ethyltestosterone, testosterone enathate, methyltestosterone, fluoxymesterone, and testosterone cypionate; estrogens such as estradiol, estropipate, and conjugated estrogens; progestins such as methoxyprogesterone acetate, and norethindrone acetate; corticosteroids such as triamcinolone, betamethasone, betamethasone sodium phosphate, dexamethasone, dexamethasone sodium phosphate, dexamethasone acetate prednisone, methylprednisolone acetate suspension, triamcinolone acetonide, methylprednisolone, prednisolone sodium phosphate, methylprednisolone sodium succinate, hydrocortisone sodium succinate, triamcinolone hexacetonide, hydrocortisone, hydrocortisone cypionate, prednisolone, fludrocortisone acetate, paramethasone acetate, prednisolone tebutate, prednisolone acetate, prednisolone sodium phosphate, and hydrocortisone sodium succinate; and thyroid hormones such as levothyroxine sodium); hypoglycemic agents (e.g., human insulin, purified beef insulin, purified pork insulin, glyburide, chlorpropamide, tolbutamide, and tolazamide); hypolipidemic agents (e.g., clofibrate, dextrothyroxine sodium, probucol, simvastatin, pravastatin, atorvastatin, lovastatin, and niacin); proteins (e.g., DNase, alginase, superoxide dismutase, and lipase); nucleic acids (e.g., sense or anti-sense nucleic acids encoding any therapeutically useful protein, including any of the proteins described herein); agents useful for erythropoiesis stimulation (e.g., erythropoietin); antiulcer/antireflux agents (e.g., famotidine, cimetidine, and ranitidine hydrochloride); antinauseants/antiemetics (e.g., meclizine hydrochloride, nabilone, prochlorperazine, dimenhydrinate, promethazine hydrochloride, thiethylperazine, and scopolamine); oil-soluble vitamins (e.g., vitamins A, D, E, K, and the like); as well as other drugs such as mitotane, halonitrosoureas, anthrocyclines, and ellipticine.

A description of these and other classes of useful drugs and a listing of species within each class can be found in Martindale, The Extra Pharmacopoeia, 30th Ed. (The Pharmaceutical Press, London 1993).

Examples of other drugs useful in the compositions and methods described herein include ceftriaxone, ceftazidime, oxaprozin, albuterol, valacyclovir, urofollitropin, famciclovir, flutamide, enalapril, fosinopril, acarbose, lorazepan, follitropin, fluoxetine, lisinopril, tramsdol, levofloxacin, zafirlukast, interferon, growth hormone, interleukin, erythropoietin, granulocyte stimulating factor, nizatidine, perindopril, erbumine, adenosine, alendronate, alprostadil, benazepril, betaxolol, bleomycin sulfate, dexfenfluramine, fentanyl, flecainid, gemcitabine, glatiramer acetate, granisetron, lamivudine, methylphenidate, mangafodipir trisodium, mesalamine, metoprolol fumarate, metronidazole, miglitol, moexipril, monteleukast, octreotide acetate, olopatadine, paricalcitol, somatropin, sumatriptan succinate, tacrine, nabumetone, trovafloxacin, dolasetron, zidovudine, finasteride, tobramycin, isradipine, tolcapone, enoxaparin, fluconazole, terbinafine, pamidronate, didanosine, cisapride, venlafaxine, troglitazone, fluvastatin, losartan, imiglucerase, donepezil, olanzapine, valsartan, fexofenadine, calcitonin, and ipratropium bromide. These drugs are generally considered to be water soluble.

Other drugs include albuterol, adapalene, doxazosin mesylate, mometasone furoate, ursodiol, amphotericin, enalapril maleate, felodipine, nefazodone hydrochloride, valrubicin, albendazole, conjugated estrogens, medroxyprogesterone acetate, nicardipine hydrochloride, zolpidem tartrate, amlodipine besylate, ethinyl estradiol, rubitecan, amlodipine besylate/benazepril hydrochloride, paroxetine hydrochloride, paclitaxel, atovaquone, felodipine, podofilox, paricalcitol, betamethasone dipropionate, fentanyl, pramipexole dihydrochloride, Vitamin D₃ and related analogues, finasteride, quetiapine fumarate, alprostadil, candesartan, cilexetil, fluconazole, ritonavir, busulfan, carbamazepine, flumazenil, risperidone, carbidopa, levodopa, ganciclovir, saquinavir, amprenavir, carboplatin, glyburide, sertraline hydrochloride, rofecoxib carvedilol, halobetasolproprionate, sildenafil citrate, celecoxib, chlorthalidone, imiquimod, simvastatin, citalopram, ciprofloxacin, irinotecan hydrochloride, sparfloxacin, efavirenz, cisapride monohydrate, lansoprazole, tamsulosin hydrochloride, mofafinil, clarithromycin, letrozole, terbinafine hydrochloride, rosiglitazone maleate, lomefloxacin hydrochloride, tirofiban hydrochloride, telmisartan, diazapam, loratadine, toremifene citrate, thalidomide, dinoprostone, mefloquine hydrochloride, chloroquine, trandolapril, docetaxel, mitoxantrone hydrochloride, tretinoin, etodolac, triamcinolone acetate, estradiol. ursodiol, nelfinavir mesylate, indinavir, beclomethasone dipropionate, oxaprozin, flutamide, famotidine, prednisone, cefuroxime, lorazepam, digoxin, lovastatin, griseofulvin, naproxen, ibuprofen, isotretinoin, tamoxifen citrate, nimodipine, amiodarone, and alprazolam.

With respect to extrusion-spheronization aids, any suitable extrusion-spheronization aids such as microcrystalline cellulose, pectin and ethylcellulose.

With respect to superdisintegrants, any superdisintegrants that can improve and modulate the release of the active pharmaceutical ingredient(s) are suitable. For example and without being limited thereto, sodium starch glycolate, sodium croscarmellose, homopolymer of cross-linked N-vinyl-2-pyrrolidone, and alginic acid, a cross-linked cellulose, a cross-linked polymer, a cross-linked starch, ion-exchange resin, crospovidone and combinations thereof.

With respect to glidants, any suitable glidant such as talc, silicon dioxide, starch, calcium silicate, Cabosil, Syloid, and silicon dioxide aerogels. Typically, silicon dioxide is used.

With respect to lubricants, any suitable lubricant are water, alkali stearates such as magnesium stearate, calcium stearate, zinc stearate, polyethylene glycol, adipic acid, hydrogenated vegetable oils, sodium chloride, sterotex, glycerol monostearate, talc, polyethylene glycol, sodium benzoate, sodium lauryl sulfate, magnesium lauryl sulfate, sodium stearyl fumarate, light mineral oil and the like may be employed. Waxy fatty acid esters, such as glyceryl behenate, sold as “Compritol” products, can be used. Other useful commercial lubricants include “Stear-O-Wet” and “Myvatex TL”. Typically, magnesium stearate, talc and/or glycerol monostearate.

With respect to oils, any suitable oil can be used, for example, one or more selected from Almond Oil, Apricot Kernel Oil, Avocado Oil, Black Currant Oil, 14% GLA, Borage Oil, 20% GLA, Canola Oil, Carrot Oil, Castor Oil, Clove Leaf Oil, Coconut Oil, Corn Oil, Cottonseed Oil, Evening Primrose Oil, 9% GLA, Flaxseed Oil, 55% ALA, Grapeseed Oil, Hazelnut Oil, Hemp Oil, ALA/GLA, Hydrogenated Oils, Jojoba Oil, Golden Jojoba Oil, Water-white Kukui Nut Oil, Macadamia Nut Oil, Oat Oil, Olive Oil, Extra Virgin Olive Oil Pomace/“B” grade, Olive Oil, Pure/NF, Palm Oil, Parsley Seed Oil, Peach Kernel Oil, Peanut Oil, Pecan Oil, Pistachio Oil, Pumpkinseed Oil, Rice Bran Oil, Rose Hip Seed Oil, Rosemary Oil, Safflower Oil, Linoleic' Safflower Oil, High-Oleic, Sesame Oil NF, Sesame Oil Toasted, Soybean Oil, Sunflower Oil, Salad Sunflower Oil High-Oleic, Tea Tree Oil, Vegetable, Glycerine, USP, Walnut Oil, Wheat Germ Oil, Cold-pressed and mineral oil or other similar oils.

With respect to a wicking agent, the wicking agent creates channels or pores. Examples include microcrystalline cellulose, pectin, colloidal silicon dioxide, kaolin, titanium dioxide, alumina, sodium lauryl sulfate, low molecular weight polyvinyl pyrrolidone, polyester and polyethylene.

With respect to electrolytes, any suitable electrolyte can be used such as one or more salts capable of providing, sodium (Na⁺), potassium (K⁺), chloride (Cl⁻), calcium (Ca²⁺), magnesium (Mg²⁺), bicarbonate (HCO₃⁻); phosphate (PO₄²⁻), and sulfate (SO₄²⁻) ions.

Examples of polymeric films include polymethacrylates copolymer and enteric materials.

With respect to an enteric material, enteric polymers useful in the present invention include esters of cellulose and its derivatives (cellulose acetate phthalate, hydroxypropyl methylcellulose phthalate, hydroxypropyl methylcellulose acetate succinate), polymethacrylates, polyvinyl acetate phthalate, methacrylic acid-methacrylate copolymers and shellac. Some commercially available materials that may be used are methacrylic acid copolymers are sold under the trademark Eudragit (L100, S100, L30D 55) manufactured by Rhom Pharma, Cellacefate (cellulose acetate phthalate) from Eastman Chemical Co., Aquateric (cellulose acetate phthalate aqueous dispersion) from FMC Corp. and hydroxypropyl methylcellulose acetate succinate aqueous dispersion from Shin Etsu K. K.

Example of non-enteric materials include cellulose ethers and ethylcellulose.

Examples of semi-permeable membrane materials includes cellulose acetate phthalate and cellulose acetate.

Examples of plasticizers include polyethylene glycol, dibutyl sebacate, triethyl citrate, castor oil, glyceryl monostearate, diethyl phthalate, and glyceryl trihepthanoate.

The term “timed release”, “pulsed release”, “chronotherapeutic release”, “extended release” and “controlled release” are defined for purposes of the present invention as the release of the drug from the dosage form at such a rate that when a dose of the drug is administered in the timed release, pulsed release, chronotherapeutic release, extended release or controlled-release form, blood (e.g., plasma) concentrations (levels) of the drug are maintained within the therapeutic range but below toxic levels over a selected period of time.

When introducing elements disclosed herein, the articles “a”, “an”, “the”, and “said” are intended to mean that there are one or more of the elements unless the context dictates otherwise. For example, the term “a compound” and “at least one compound” may include a plurality of compounds, including mixtures thereof. The terms “comprising”, “having”, “including” are intended to be open-ended and mean that there may be additional elements other than the listed elements.

The above disclosure generally describes the present invention. A more complete understanding can be obtained by reference to the following specific Examples. The Examples are described solely for purposes of illustration and are not intended to limit the scope of the invention. Changes in form and substitution of equivalents are contemplated as circumstances may suggest or render expedient. Although specific terms have been employed herein, such terms are intended in a descriptive sense and not for purposes of limitation.

### EXAMPLES

### Example 1

### Controlled Release Methylphenidate HCl Spheroids

This was a two step process in which immediate release spheroids were manufactured by an extrusion-spheronization process followed by application of a controlled release coating on the spheroids to form controlled release spheroids.

(1) Manufacture of Spheroid without Coating

| | Formulation I | Formulation II | Formulation III |
|---|---|---|---|
| Components | (wt %) | (wt %) | (wt %) |
| Methylphenidate HCl | 25 | 25 | 20 |
| Carbomer | 0.5 | — | — |
| Pectin | 5 | — | — |
| Microcrystalline | 60 | 60 | 60 to 67 |
| cellulose | | | |
| Ethylcellulose* | — | — | 3 to 10 |
| Crospovidone | 4.5 | 5 | 5 |
| Talc | 5 | 10 | 5 |
| Water | QS | QS | QS |

*Used as aqueous granulating solution (Aquacoat ™)
QS was typically about 100 wt % to about 200 wt %

With respect to each formulation, the materials were charged into a planetary mixer and blended for about 5 minutes. The resultant homogeneous blend was granulated for about 3 minutes with the sufficient quantity of water with respect to Formulation I and Formulation II, while an aqueous suspension of ethylcellulose (commercial brand Aquacoat™) was used for Formulation III. The wet mass was extruded using a Caleva extruder Model 25. The extrudates were spheronised in about 500 gram quantities in a Caleva spheroniser Model 240. The wet spheroids were dried at about 40° C. in a tray dryer oven to LOD (loss on drying) of less than about 2 wt %.

### (2) Coating of Spheroid

About 1000 g of the spheroids from Formulation I were coated with an aqueous dispersion composed of about 500 g of Aquacoat™ (e.g. ethylcellulose dispersion), about 40 g LustreClear™ (e.g. carrageenan and microcrystalline cellulose), about 35.5 g of dibutyl sabate, and about 114 g of water. The spheroids were coated to a weight gain of about 6% of the spheroid weight.

About 1000 g of the spheroids from Formulation II were coated with an aqueous dispersion composed of about 500 g of Aquacoat™ (e.g. ethylcellulose dispersion), about 40 g LustreClear™ (e.g. carrageenan and microcrystalline cellulose), about 36 g of dibutyl sabate, and about 114 g of water. The spheroids were coated to a weight gain of about 6% of the spheroid weight.

About 1250 g of the spheroids from Formulation III were coated with an aqueous dispersion composed of about 350 g of Aquacoat™ (e.g. ethylcellulose dispersion), about 36 g of dibutyl sabate, 20 g of pigment and about 72 g of water. The spheroids were coated to a weight gain of about 12% of the spheroid weight.

Coating was done in a UniGlatt fluid bed coater using a top spray assembly. The coated spheroids were dried in a tray dryer oven for about 2 hours at about 60° C.

### Example 2

### Pulsed Release Venlafaxine HCl Capsules or Tablets

This was a three step process in which immediate release spheroids were manufactured by an extrusion-spheronization process followed by application of a controlled release coat on some of the spheroids. To obtain pulsed release, a coated population of spheroids were combined with an uncoated population of spheroids and encapsulated in a capsule or compressed into a tablet. Alternatively, coated spheroids with different release rates were combined together and encapsulated in a capsule or compressed into a tablet.

### (1) Manufacture of Immediate Release Spheroids

| | Formulation IV | Formulation V |
|---|---|---|
| Components | (wt %) | (wt %) |
| Venlafaxine HCl | 39 | 40 |
| Pectin | 5 | — |
| Microcrystalline cellulose | 45 | 45 |
| Sodium chloride | — | 2 |
| Coconut Oil | 1 | — |
| Crospovidone | 5 | 3 |
| Talc | 5 | 10 |
| Water | QS | QS |

QS was typically about 100 wt % to about 200 wt %

With respect to each formulation, the materials were charged into a planetary mixer and blended for about 5 minutes. The resultant homogeneous blend was granulated for about 3 minutes with the sufficient quantity of water. The wet mass was extruded using a Caleva extruder Model 25. The extrudates were spheronised in about 500 gram quantities in a Caleva spheroniser Model 240. The wet spheroids were dried at about 40° C. in a tray dryer oven to LOD (loss on drying) of less than about 2 wt %.

### (2) Coating of Immediate Release Spheroids

About 1000 g of the spheroids from Formulation IV were coated with an aqueous dispersion composed of about 500 g of Aquacoat™ (e.g. ethylcellulose dispersion), about 40 g LustreClear™ (e.g. carrageenan and microcrystalline cellulose), about 36 g of dibutyl sabate, and about 114 g of water. The spheroids were coated to a weight gain of about 6% of the spheroid weight to yield Formulation IVa, while Formulation V was coated to a weight gain of 15% of the spheroid weight using a similar aqueous dispersion to yield Formulation Va.

Coating was done in a UniGlatt fluid bed coater using a top spray assembly. The coated spheroids were dried in a tray dryer oven for about 2 hours at about 60° C.

### (3) Assembly of Pulsed Release Venlafaxine HCl

### Type 1

Type 1 is made of a blend of 10 wt % Formulation IV, 45 wt % Formulation IVa and 45 wt % Formulation Va.

### Type 2

Type 2 is made of a blend of 30 wt % Formulation IV, and 70 wt % Formulation Va.

### Type 3

Type 3 is made of a blend of 40 wt % Formulation IVa and 60 wt % Formulation Va.
These combinations (Type 1, Type 2 or Type 3) were encapsulated or compressed into tablets.

### Example 3

### Chronotherapeutic or Timed Release Carvedilol Capsules or Tablets

This was a three step process in which immediate release spheroids were manufactured by a solution layering process in a fluid bed coater followed by application of a controlled release coat on the spheroids. To obtain chronotherapeutic release, a controlled release coated population of spheroids were coated with methacrylic acid copolymer and/or cellulose esters and encapsulated in a capsule. Alternatively, a controlled release coated population of spheroids were compressed into a tablet and the tablet was coated with methacrylic acid copolymer and/or cellulose esters.

### (1) Manufacture of Immediate Release Spheroids

| | Formulation VI | Formulation VII | Formulation VIII |
|---|---|---|---|
| Components | (wt %) | (wt %) | (wt %) |
| Carvedilol | 5 | 5 | 5 |
| Extruded Sugar | 88 | 88 | 88 |
| spheres | | | |
| *LustreClear ™ | 5 | — | 2 |
| **Opadry ™ | — | 5 | 3 |
| Crospovidone | 2 | 2 | 2 |
| Water | QS | QS | QS |

*contain carrageenan and microcrystalline cellulose
**contain hydroxypropylmethyl cellulose
QS was typically about 100 wt % to about 200 wt %

With respect to each formulation, Carvedilol and crospovidone were slowly added to an aqueous solution of LustreClear™ and/or Opadry™ and mixed well. Sugar spheres (18-20 mesh) were coated with the drug suspension in a UniGlatt fluid bed coater. The spheroids were coated to a weight gain of about 10% of the spheroid weight. The spheroids were dried to LOD (loss on drying) of less than about 2 wt %.

### (2) Manufacture of Controlled Release Spheroids

About 1000 g of the spheroids from Formulation VI were coated with an aqueous dispersion composed of about 500 g of Aquacoat™ (e.g. ethylcellulose dispersion), about 40 g LustreClear™ (e.g. carrageenan and microcrystalline cellulose), about 35.5 g of dibutyl sabate, and about 114 g of water. The spheroids were coated to a weight gain of about 6% of the spheroid weight.

About 1000 g of the spheroids from Formulation VII were coated with an aqueous dispersion composed of about 500 g of Aquacoat™ (e.g. ethylcellulose dispersion), about 40 g LustreClear™ (e.g. carrageenan and microcrystalline cellulose), about 36 g of dibutyl sabate, and about 114 g of water. The spheroids were coated to a weight gain of about 6% of the spheroid weight.

About 1000 g of the spheroids from Formulation VIII were coated with an aqueous dispersion composed of about 500 g of Aquacoat™ (e.g. ethylcellulose dispersion), about 40 g LustreClear™ (e.g. carrageenan and microcrystalline cellulose), about 36 g of dibutyl sabate, and about 114 g of water. The spheroids were coated to a weight gain of about 6% of the spheroid weight.

About 1000 g of the spheroids from Formulation VI were coated with an aqueous dispersion composed of about 400 g of Eudragit NE30D™ and about 60 g of talc to a weight gain of about 6% of the spheroid weight.

About 1000 g of the spheroids from Formulation VII were coated with an aqueous dispersion composed of about 400 g of Eudragit NE30D™ and about 60 g of talc to a weight gain of about 6% of the spheroid weight.

About 1000 g of the spheroids from Formulation VIII were coated with an aqueous dispersion composed of about 400 g of Eudragit NE30D™ and about

60 g of talc to a weight gain of about 6% of the spheroid weight.

Coating was done in a UniGlatt fluid bed coater using a top spray assembly. The coated spheroids were dried in a tray dryer oven for about 2 hours at about 60° C.

### (3) Manufacture of Chronotherapeutic or Timed Release Carvedilol

### (I) Capsules

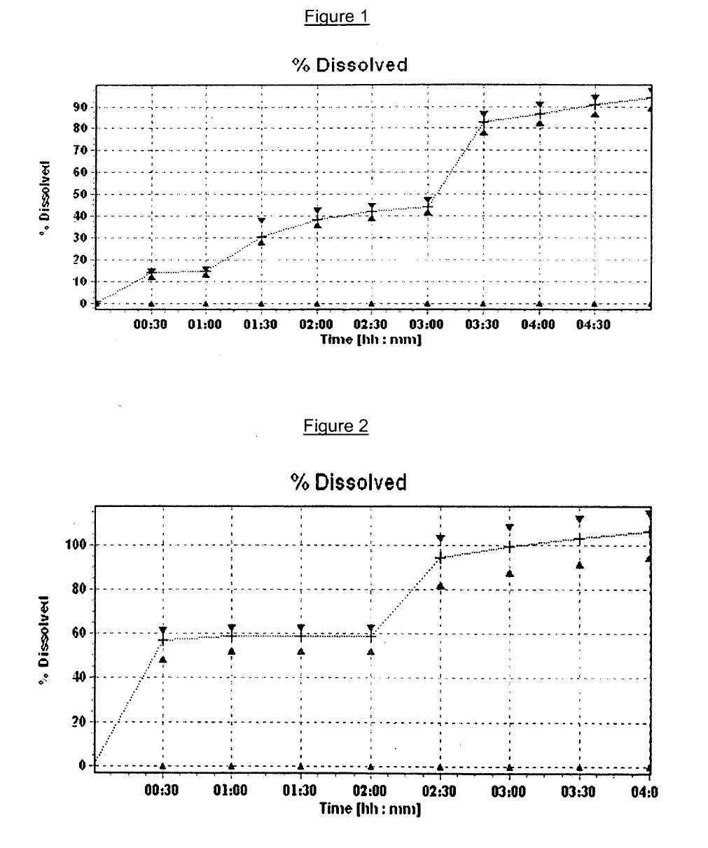

The controlled release spheroids were coated with an aqueous dispersion composed of about 1142 g of Eudragit L₃₀D55™ (e.g. methacrylic acid copolymer), about 137 g of glycerol monostearate, about 41 g of triacetyl citrate, and about 679 g of water and/or an aqueous dispersion composed of about 1142 g of cellulose esters, about 137 g of glycerol monostearate, about 41 g of triacetyl citrate, and about 679 g of water to a weight gain sufficient to give a lag time of about 1 hour to about 12 hours as desired. These are then encapsulated in a capsule. FIG. 1 shows a dissolution profile for these capsules.

### (II) Tablets

The controlled release coated population of spheroids and/or inert spheroids were compressed into a tablet and the tablet was coated an aqueous dispersion composed of about 1142 g of Eudragit L30D₅₅TM (e.g. methacrylic acid copolymer), about 137 g of glycerol monostearate, about 41 g of triacetyl citrate, and about 679 g of water and/or an aqueous dispersion composed of about 1142 g of cellulose esters, about 137 g of glycerol monostearate, about 41 g of triacetyl citrate, and about 679 g of water to a weight gain sufficient to give a lag time of about 1 hour to about 12 hours as desired. FIG. 2 shows a dissolution profile for these tablets.

1. A drug delivery composition comprising extruded spheroids, the spheroids comprising:
at least one active pharmaceutical ingredient; at least one extrusion-spheronization aid; at least one superdisintegrant; and at least one glidant, at least one lubricant, and/or at least one oil.

2. The drug delivery composition of claim 1, wherein said at least one active pharmaceutical ingredient is from about 0.1 wt % to about 80 wt %.

3. The drug delivery composition of claim 1, wherein said at least one active pharmaceutical ingredient is from about 5 wt % to about 70 wt %; about 10 wt % to about 70 wt %; about 20 wt % to about 60 wt %; about 30 wt % to about 60 wt %; or from about 40 wt % to about 60 wt %.

4. The drug delivery composition of claim 1, wherein said at least one extrusion-spheronization aid is from about 10 wt % to about 70 wt %; from about 20 wt % to about 70 wt %; about 30 wt % to about 70 wt %; about 40 wt % to about 70 wt %; about 50 wt % to about 70 wt %; or from about 55 wt % to about 70 wt %.

5. The drug delivery composition of claim 1, wherein said at least one superdisintegrant is from about 2 wt % to about 70 wt %; from about 20 wt % to about 70 wt %; about 30 wt % to about 70 wt %; about 40 wt % to about 70 wt %; about 50 wt % to about 70 wt %; or from about 55 wt % to about 70 wt %.

6. The drug delivery composition of claim 1, wherein said at least one glidant is from about 1 wt % to about 20 wt %; from about 1 wt % to about 15 wt %; from about 2 wt % to about 15 wt %; from about 5 wt % to about 15 wt %; or from about 5 wt % to about 10 wt %.

7. The drug delivery composition of claim 1, wherein said at least one lubricant is from about 0.5 wt % to about 5 wt %; from about 0.5 wt % to about 4 wt %; from about 0.5 wt % to about 3 wt %; from about 0.5 wt % to about 2 wt %; or from about 1 wt % to about 2 wt %.

8. The drug delivery composition of claim 1, wherein said at least one oil is from about 0.5 wt % to about 5 wt %; from about 0.5 wt % to about 4 wt %; from about 0.5 wt % to about 3 wt %; from about 0.5 wt % to about 2 wt %; or from about 1 wt % to about 2 wt %.

9. The drug delivery composition of claim 1, further comprising at least one carbomer, at least one buffering agent, at least one electrolyte, zein, and/or water.

10. The drug delivery composition of claim 1, wherein said at least one extrusion-spheronization aid is from about 10 wt % to about 90 wt %, said at least one superdisintegrant is from about 0.1 wt % to about 70 wt %, said at least one glidant is from about 0.1 wt % to about 70 wt %, said at least one lubricant is from about 0.1 wt % to about 70 wt % and said at least one oil is from about 0.1 wt % to about 50 wt %.

11. The drug delivery composition of claim 1, further comprises at least one coating.

12. The drug delivery composition of claim 11, wherein said at least one coating comprises at least one layer of a polymeric film coat; at least one layer of an enteric coat; at least one layer of a non-enteric coat; and/or at least one layer of a semi-permeable membrane coat.

13. The drug delivery composition of claim 12, wherein said at least one layer of an enteric coating comprises at least one enteric material and at least one superdisintegrant; said at least one layer of a non-enteric coat comprises at least one non-enteric material and at least one superdisintegrant; and/or said at least one layer of a semi-permeable membrane coat comprises at least one semi-permeable membrane material and at least one superdisintegrant.

14. The drug delivery composition of claim 13, wherein said at least one superdisintegrant is from about 0.5 wt % to about 55 wt %; from about 0.5 wt % to about 40 wt %; from about 0.5 wt % to about 30 wt %; from about 1 wt % to about 20 wt %; or from about 10 wt % to about 20 wt %.

15. The drug delivery composition of claim 12, wherein each layer further comprises at least one wicking agent, carragenaan, and at least one plasticizer.

16. The drug delivery composition of claim 15, wherein said at least one wicking agent is from about 0.5 wt % to about 55 wt %; from about 0.5 wt % to about 50 wt %; from about 0.5 wt % to about 40 wt %; from about 5 wt % to about 40 wt %; or from about 20 wt % to about 40 wt %.

17. The drug delivery composition of claim 15, wherein carragenaan is from about 0.5 wt % to about 55 wt %; from about 0.5 wt % to about 50 wt %; from about 0.5 wt % to about 40 wt %; from about 5 wt % to about 40 wt %; or from about 20 wt % to about 40 wt %.

18. The drug delivery composition of claim 15, wherein the plasticizer is from about 0.5 wt % to about 25 wt %; from about 1 wt % to about 20 wt %; from about 5 wt % to about 20 wt %; from about 5 wt % to about 15 wt %; or from about 1 wt % to about 5 wt %.

19. The drug delivery composition of claim 11, wherein said at least one coating from about 0.5 wt % to about 50 wt % based on the total weight of the spheroid and coating.

20. The drug delivery composition of claim 19, wherein said at least one coating is from about I wt % to about 20 wt %, from about I wt % to about 10 wt %, from about 1 wt % to about 7 wt %, from about 3.5 wt % to about 7 wt %, from about 3.5 wt % to about 6 wt %, or from about 4 wt % to about 5 wt %.

21. The drug delivery composition of claim 19, wherein the coating is applied to the spheroids to yield a surface area of about 0.1 mg/cm² to about 20 mg/cm².

22. The drug delivery composition of claim 1, wherein the drug delivery composition is encapsulated or compressed into a tablet and/or caplet.

23. The drug delivery composition of claim 1, wherein the spheroids are in a capsule and/or sachet.

24. The drug delivery composition of claim 1, wherein there are different populations of the spheroids.

25. The drug delivery composition of claim 1, wherein said at least one active pharmaceutical ingredient comprises chemical or biological molecules providing a therapeutic, diagnostic, or prophylactic effect in vivo.

26. The drug delivery composition of claim 1, wherein at least one extrusion-spheronization aid comprises microcrystalline cellulose, pectin and/or ethylcellulose.

27. The drug delivery composition of claim 1, wherein said at least one superdisintegrants comprises sodium starch glycolate, sodium croscarmellose, homopolymer of cross-linked N-vinyl-2-pyrrolidone, and alginic acid, a cross-linked cellulose, a cross-linked polymer, a cross-linked starch, ion-exchange resin, and/or crospovidone.

28. The drug delivery composition of claim 1, wherein said at least one glidant comprises silicon dioxide, starch, calcium silicate, talc, Cabosil, Syloid, and silicon dioxide aerogels.

29. The drug delivery composition of claim 1, wherein said at least one lubricant comprises alkali stearate, polyethylene glycol, adipic acid, hydrogenated vegetable oil, sodium chloride, sterotex, glycerol monostearate, talc, polyethylene glycol, sodium benzoate, sodium lauryl sulfate, magnesium lauryl sulfate, sodium stearyl fumarate, light mineral oil, and/or waxy fatty acid ester.

30. The drug delivery composition of claim 1, wherein said at least one oil comprises one or more selected from Almond Oil, Apricot Kernel Oil, Avocado Oil, Black Currant Oil, 14% GLA, Borage Oil, 20% GLA, Canola Oil, Carrot Oil, Castor Oil, Clove Leaf Oil, Coconut Oil, Corn Oil, Cottonseed Oil, Evening Primrose Oil, 9% GLA, Flaxseed Oil, 55% ALA, Grapeseed Oil, Hazelnut Oil, Hemp Oil, ALA/GLA, Hydrogenated Oils, Jojoba Oil, Golden Jojoba Oil, Water-white Kukui Nut Oil, Macadamia Nut Oil, Oat Oil, Olive Oil, Extra Virgin Olive Oil Pomace/“B” grade, Olive Oil, Pure/NF, Palm Oil, Parsley Seed Oil, Peach Kernel Oil, Peanut Oil, Pecan Oil, Pistachio Oil, Pumpkinseed Oil, Rice Bran Oil, Rose Hip Seed Oil, Rosemary Oil, Safflower Oil, Linoleic' Safflower Oil, High-Oleic, Sesame Oil NF, Sesame Oil Toasted, Soybean Oil, Sunflower Oil, Salad Sunflower Oil High-Oleic, Tea Tree Oil, Vegetable, Glycerine, USP, Walnut Oil, Wheat Germ Oil, Cold-pressed and mineral oil or other similar oils.

31. A drug delivery composition comprising coated spheroids having inert spheroids and at least one coating for the spheroids, the coating comprising at least one active pharmaceutical ingredient and at least one superdisintegrant.

32. The drug delivery composition of claim 31, wherein said at least one active pharmaceutical ingredient is from about 0.1 wt % to about 90 wt %.

33. The drug delivery composition of claim 31, wherein
said at least one
superdisintegrant is from about 0.1 wt % to about 80 wt %.

34. The drug delivery composition of claim 31, wherein said at least one active pharmaceutical ingredient is from about 0.1 wt % to about 80 wt %; from about 10 wt % to about 80 wt %; from about 20 wt % to about 80 wt %; from about 30 wt % to about 70 wt %; or from about 40 wt % to about 70 wt %.

35. The drug delivery composition of claim 31, wherein said at least one superdisintegrant is from about 0.5 wt % to about 55 wt %; from about 0.5 wt % to about 40 wt %; from about 0.5 wt % to about 30 wt %; from about 1 wt % to about 20 wt %; or from about 10 wt % to about 20 wt %.

36. The drug delivery composition of claim 31, wherein the coating further comprises at least one of a wicking agent, carageenan and/or a plasticizer.

37. The drug delivery composition of claim 36, the wicking agent is from about 0.5 wt % to about 90 wt %; from about 0.5 wt % to about 80 wt %; from about 0.5 wt % to about 70 wt %; from about 5 wt % to about 60 wt %; or from about 20 wt % to about 60 wt %.

38. The drug delivery composition of claim 36, the carragenaan is from about 0.5 wt % to about 90 wt %; from about 0.5 wt % to about 80 wt %; from about 0.5 wt % to about 70 wt %; from about 5 wt % to about 60 wt %; or from about 20 wt % to about 60 wt %.

39. The drug delivery composition of claim 36, wherein the plasticizer is from about 0.5 wt % to about 25 wt %; from about 1 wt % to about 20 wt %; from about 5 wt % to about 20 wt %; from about 5 wt % to about 15 wt %; or from about 1 wt % to about 5 wt %.

40. The drug delivery composition of claim 31, wherein said at least one coating comprises from about 0.1 wt % to about 80 wt % of said at least one active pharmaceutical ingredient, from about 0.1 wt % to about 50 wt % of said at least one superdisintegrant, from about 0.5 wt % to about 90 wt % of a wicking agent, and from about 0.5 wt % to about 90 wt % of carrageenan.

41. The drug delivery composition of claim 31, wherein said at least one active pharmaceutical ingredient comprises chemical or biological molecules providing a therapeutic, diagnostic, or prophylactic effect in vivo.

42. The drug delivery composition of claim 31, wherein said at least one superdisintegrants comprises sodium starch glycolate, sodium croscarmellose, homopolymer of cross-linked N-vinyl-2-pyrrolidone, and alginic acid, a cross-linked cellulose, a cross-linked polymer, a cross-linked starch, ion-exchange resin, and/or crospovidone.

43. The drug delivery composition of claim 31, wherein said spheroids comprise said at least one coating from about 0.5 wt % to about 50 wt % based on the total weight of the spheroid and coating.

44. The drug delivery composition of claim 43, wherein said at least one coating is from about 1 wt % to about 20 wt %, from about 1 wt % to about 10 wt %, from about 1 wt % to about 7 wt %, from about 3.5 wt % to about 7 wt %, from about 3.5 wt % to about 6 wt %, or from about 4 wt % to about 5 wt %.

45. The drug delivery composition of claim 43, wherein said at least one coating is applied to the spheroids to yield a surface area of about 0.1 mg/cm² to about 20 mg/cm².

46. The drug delivery composition of claim 31, wherein the drug delivery composition is encapsulated or compressed into a tablet and/or caplet.

47. The drug delivery composition of claim 31, wherein the spheroids are in a capsule and/or sachet.

48. The drug delivery composition of claim 31,
wherein
there are different populations of spheroids.

49. The drug delivery composition of claim 31, wherein the inert spheroids are any pharmaceutically acceptable, inert spheroids.

50. The drug delivery composition of claim 49, wherein the inert spheroids comprise sugar spheroids, starch spheroids and/or cellulose spheroids.

51. A method for administering the drug delivery composition of claim 1 or 31 to a mammal to provide a timed, pulsed, chronotherapeutic, extended or controlled release of said at least one active pharmaceutical ingredient.

52. The method of claim 51 for treating a disease for which said at least one active pharmaceutical ingredient in the drug delivery composition is effective.

53-56. (canceled)

57. A method for making the drug delivery composition of claim 1, the method comprising:
combining dry materials of the composition to provide a homogeneous blend;
combining the granules with said at least one glidant, at least one lubricant, and/or at least one oil to provide a wetted mass suitable for extrusion-spheronization; and
extruding the wetted mass to form the spheroids.

58. The method of claim 57, wherein the wetted mass has a plasticity.

59. The method of claim 57, wherein the wetted mass comprises from about 1:0.7 to about 1:2 of the extrusion aid to said at least one glidant, at least one lubricant, and/or at least one oil.

60. The method of claim 57, wherein the granules are further combined with at least one plasticizer.

61. The method of claim 57, wherein after extruding the wetted mass, the extrudates are charged onto a spheronizer rotating plate and spun to provide the spheroids.

62. The method of claim 57, wherein the spheroids are dried to provide spheroids having a water content of less than about 10 wt %.

63. The method of claim 57 further comprising coating the spheroids.

64-66. (canceled)
